(19) United States
(12) Patent Application Publication
(10) Pub. No.: US 2007/0142821 A1
(43) Pub. Date: Jun. 21, 2007

(54) Rapid exchange catheter having a uniform diameter exchange joint

(75) Inventors: Joe Hennessy, Ballybrit (IE); Ashish Varma, Ballybirt (IE); Robert Murray, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/303,755

(22) Filed: Dec. 16, 2005

(51) Int. Cl. A61M 25/00

(52) U.S. Cl. 604/528

(57) ABSTRACT

A rapid exchange catheter includes an elongate proximal shaft comprising a proximal lumen, an elongate distal shaft defining an distal outer lumen, an elongate distal inner lumen inside the distal shaft, and an exchange joint coupled between the elongate proximal shaft and the elongate distal shaft. The exchange joint includes a guidewire port providing external access for a guidewire into the elongate distal inner lumen, and a transition lumen in communication with the proximal lumen and the distal outer lumen. The transition lumen has a crescent-shaped cross-section at least at the exchange joint distal end.

### TECHNICAL FIELD

The present invention generally relates to catheters used in the vascular system, and more particularly relates to systems for facilitating exchange of such catheters and associated guidewires, and for using such catheters and guidewires to access selected sites within a patient.

### BACKGROUND

Catheters are inserted into various locations within a patient for a wide variety of purposes and medical procedures. Catheter insertion typically requires the use of a guidewire, particularly when the catheter carries a stent or other relatively bulky therapeutic device. The guidewire may be inserted into a patient's vasculature through the skin, and advanced to the treatment location. Alternatively, the guidewire and the delivery catheter may be advanced together, with the guidewire protruding from the catheter distal end. In either case, the guidewire guides the delivery catheter to the treatment location.

There are various types of catheters, one of which is the “rapid exchange” (RX) or single operator catheter, which is formed with a relatively short guidewire lumen that extends through a short distal catheter segment. The guidewire proximal exit port is typically located about 5 cm to about 30 cm from the catheter distal end. During use, the guidewire is initially placed in the patient's vascular system, and the catheter distal segment is then threaded onto the guidewire. The catheter can be advanced alongside the guidewire with its distal segment being attached to and guided along the guidewire. The catheter can be removed and exchanged for another RX catheter without the need for a relatively long exchange guidewire and without withdrawing the initially placed guidewire.

A cross sectional longitudinal view of one type of RX catheter 50 is depicted in FIG. 1. The RX catheter 50 includes an elongate distal shaft 56 joined to transition tubing 52. The distal shaft 56 includes a coaxial inner guidewire lumen 54 extending to the shaft distal end 53. The transition tubing 52 joins the distal shaft 56 to a proximal shaft 51, which may include or function as an inflation lumen through which a fluid is transported to inflate a balloon 55 when a therapeutic procedure is performed using the RX catheter 50. FIG. 2 is a cross-sectional longitudinal view of an exchange joint 60 where the distal shaft 56 and the transition tubing 52 are joined. As depicted, the transition tubing 52 is inserted into the distal shaft 56. The guidewire lumen 54 is situated alongside the transition tubing at the position where the transition tubing 52 is inserted. During use, the transition tubing 52 transports fluid from the proximal shaft 51 to a distal shaft inflation lumen 57 that is coaxial with the guidewire lumen 54. Thus, the exchange joint 60 effectively transitions the inflation and guidewire lumens into the distal shaft 56 from a proximal side-by-side arrangement to a distal coaxial arrangement.

Assembly of the exchange joint 60 is a somewhat intricate and inefficient process because of the number of components that are bonded together. The assembly process includes flaring the inner diameter of the distal shaft 56 to allow room for insertion of the transition tubing 52, which also may require skiving to minimize the space taken by the transition tubing 52 inside the distal shaft 56. At some point prior to bonding, mandrels are inserted into the guidewire lumen 54 and into the transition tubing 52 in order to prevent their respective passageways from collapsing. FIGS. 3 and 6 are cross sectional views of the exchange joint 60 taken along line A-A in FIG. 2 before and after performing a bonding procedure, with a D mandrel 59 loaded into the transition tubing and a round mandrel loaded into the guidewire lumen 54. The bonding process includes wrapping heat shrink material around the exchange joint 60. Heat is then applied to the exchange joint 60 as the heat shrink material compresses the joint components and brings the joint 60 to the bonded form depicted in FIG. 4.

In addition to its inherently intricate assembly process, the formed exchange joint 60 gives the overall RX catheter a distinctively stepped shape as seen when viewing the joint 60 in FIG. 2. The step in the exchange joint 60 adds bulk to the shaft diameter, which may detrimentally affect catheter performance. More particularly, if the RX catheter 50 exits the distal end of a guide catheter, the step in the exchange joint 60 may become caught on the guide catheter edge while withdrawing the RX catheter from the vessel.

Recent improvements to RX catheters have simplified their exchange joints. For example, FIG. 5 is a cross-sectional perspective view of an exchange joint 70 disclosed in International Publication No. WO 2005/021080. The joint 70 is a unitary molded structure that includes a guidewire port 62 through which a guidewire is introduced into a guidewire lumen 64 inside a distal shaft 66. The joint 70 is tailored at its proximal end 65 for bonding to a proximal shaft 68, and is further tailored at its distal end for bonding to the distal shaft 66. The guidewire port 62 is also tailored for bonding to the guidewire lumen 64 in a manner that produces a side-by-side arrangement between the guidewire lumen 64 and an inflation lumen 69 in the distal shaft 66. Although the molded joint 70 greatly simplifies the overall exchange joint construction, the side-by-side arrangement of the guidewire lumen 64 and the inflation lumen 69 produces a relatively bulky distal shaft 66. Further, the molded joint proximal end 65 is formed around the outer surface of the proximal shaft 68, producing a step that may become caught on a guide catheter edge while withdrawing the catheter from the vessel.

Accordingly, it is desirable to provide an RX catheter that includes an exchange joint that has a comparatively low profile and a substantially uniform outer diameter throughout the joint and at interfaces between the joint and the lumens that the joint brings together. In addition, it is desirable to provide an RX catheter that is simple and efficient to assemble. Furthermore, other desirable features and characteristics of the present invention will become apparent from the subsequent detailed description and the appended claims, taken in conjunction with the accompanying drawings and the foregoing technical field and background.

### BRIEF SUMMARY

According to one embodiment of the invention, a rapid exchange catheter is provided. The catheter includes an elongate proximal shaft comprising a proximal lumen, an elongate distal shaft defining an distal outer lumen, an elongate distal inner lumen inside the distal shaft, and an exchange joint coupled between the elongate proximal shaft and the elongate distal shaft. The exchange joint includes a guidewire port providing external access for a guidewire into the elongate distal inner lumen, and a transition lumen in communication with the proximal lumen and the distal outer lumen. The transition lumen has a crescent-shaped cross-section at least at the exchange joint distal end.

According to another embodiment of the invention, an exchange joint is provided for joining a proximal shaft, a distal shaft, and an elongate distal inner lumen inside the distal shaft in a rapid exchange catheter. The exchange joint includes a proximal end adapted to be coupled to the proximal shaft, a distal end adapted to be coupled to the distal shaft, a guidewire port providing external access for a guidewire into the elongate distal inner lumen, and a transition lumen in communication with the proximal shaft and the distal shaft. The transition lumen has a substantially round cross section at the exchange joint proximal end, and a crescent-shaped cross-section at least at the exchange joint distal end.

### BRIEF DESCRIPTION OF THE DRAWINGS

The present invention will hereinafter be described in conjunction with the following drawing figures, wherein like numerals denote like elements, and

FIG. 1 is a cross-sectional longitudinal view of an RX catheter;

FIG. 2 is a cross-sectional longitudinal view of an exchange joint from the RX catheter depicted in FIG. 1;

FIG. 3 is a cross-sectional view of the exchange joint depicted in FIG. 1 before performing a bonding step, the view taken along line 5-5;

FIG. 4 is a cross-sectional view of the exchange joint depicted in FIG. 3 after performing a bonding step;

FIG. 5 is a cross-sectional perspective view of a molded exchange joint in an RX catheter;

FIG. 6 is a perspective view of a unitary exchange joint for an RX catheter according to an embodiment of the invention;

FIG. 7 is an end view of the distal end of the unitary exchange joint depicted in FIG. 6;

FIG. 8 is an end view of the proximal end of the unitary exchange joint depicted in FIG. 7;

FIG. 9 is a perspective view of a unitary exchange joint for an RX catheter according to another embodiment of the invention;

FIG. 10 is a cross-sectional longitudinal view of the unitary exchange joint depicted in FIG. 9, in conjunction with an RX catheter proximal shaft and distal shaft, and further in conjunction with a guidewire lumen;

FIG. 10 is a perspective view of the unitary exchange joint depicted in FIG. 9, the view taken from the joint distal end;

FIG. 12 is a perspective view of the unitary exchange joint depicted in FIG. 9, the view taken from the joint proximal end;

FIG. 13 is a perspective view of a stepped crescent-shaped mandrel according to an embodiment of the invention;

FIG. 14 is a cross-sectional longitudinal view of a multi-component RX catheter exchange joint, including a proximal shaft, a distal shaft, a hypotube functioning as an inflation lumen, a guidewire lumen, and mandrels that are inserted into the inflation lumen and the guidewire lumen during a bonding process; and

FIG. 15 is a cross-sectional longitudinal view of a unitary RX catheter exchange joint, a nanotube that functions as a guidewire lumen, a distal shaft, and a proximal shaft depicted to illustrate their relative configuration for a bonding assembly using a pair of illustrated mandrels; and

FIG. 16 is a cross-sectional view of an exchange joint in an RX catheter after performing a bonding procedure, the exchange joint including mandrels in the guidewire lumen and the inflation lumen.

### DETAILED DESCRIPTION

The following detailed description is merely exemplary in nature and is not intended to limit the invention or the application and uses of the invention. Furthermore, there is no intention to be bound by any expressed or implied theory presented in the preceding technical field, background, brief summary or the following detailed description.

The present invention includes an RX catheter having an exchange joint in the catheter distal region. The exchange joint may either be a unitary structure or a combination of components as in the previously-described examples, and may have a substantially uniform outer diameter due to a compact arrangement of a plurality of lumens. The RX catheter is also efficiently assembled during a catheter assembly procedure using the exchange joint.

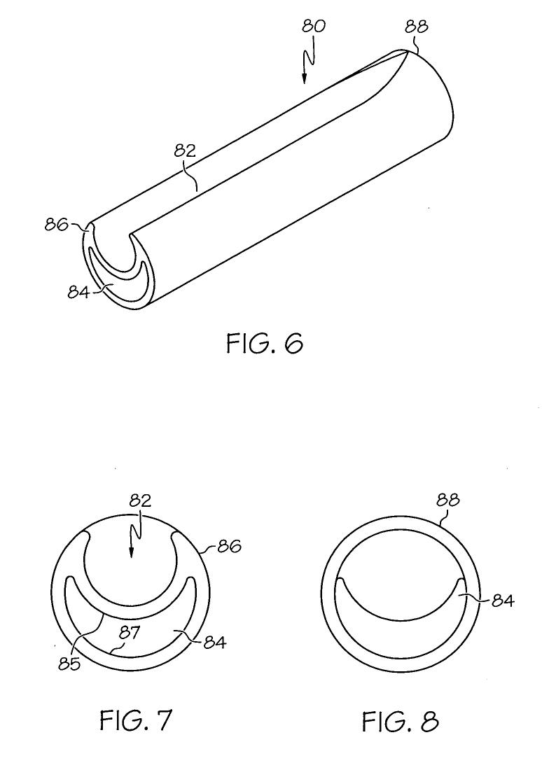

FIG. 6 is a perspective view of an exemplary unitary exchange joint 80 having a substantially uniform outer diameter. FIGS. 7 and 8 are end views of the exchange joint 80 from the joint distal and proximal ends 86 and 88, respectively. The exchange joint 80 includes a guidewire port 82 that provides external access to a guidewire lumen in an RX catheter distal shaft. A guidewire is directed into the guidewire lumen by inserting the guidewire into the guidewire port 82 and feeding it through the exchange joint 80. The guidewire port 82 begins proximate to the exchange joint proximal end 88 and gradually forms a deepening trench in the exchange joint outer surface. At the exchange joint distal end 86, the guidewire port 82 almost entirely encloses a guidewire, and the wall defining the guidewire port consequently has a C-shaped, nearly circular cross-section. An inflation lumen 84 is also included in the exchange joint, and transitions from a substantially circular proximal cross sectional shape to a substantially crescent-shaped distal cross sectional shape. Since the exchange joint is a molded structure, it can be mass manufactured. Further, the unitary joint structure enables quick assembly of an RX catheter.

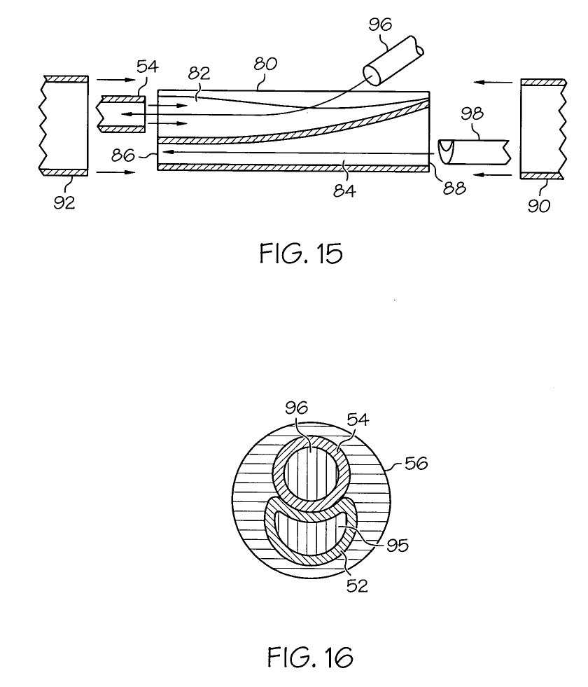

Turning to FIG. 15, a cross-sectional longitudinal view of the exchange joint 80, a guidewire lumen 54, a distal shaft 92, and a proximal shaft 90 are depicted to illustrate their relative configuration for a bonding assembly using a pair of mandrels 96, 98. The proximal end of the guidewire lumen 54 is inserted into the guidewire port 82, and a wire mandrel 96 is inserted into the guidewire lumen 54 to prevent the hypotube from collapsing when the exchange joint components are bonded. For the same reason, a crescent-shaped mandrel 98 is inserted into the inflation lumen 84. With the guidewire lumen 54 and the mandrels 96 and 98 in place, the distal shaft 92 is slid around the exchange joint distal end 86 and heat shrink is wrapped around the joined components. The assembly is then heated, and the heat coupled with compression force from the heat shrink bonds the exchange joint distal end 86 to the distal shaft 92 and to the guidewire lumen 54. The mandrels are removed, and the exchange joint distal end 88 is then inserted into the proximal shaft 90. The exchange joint 80 and the proximal shaft 90 are bonded using heat and compression force from heat shrink wrapped around the assembly.

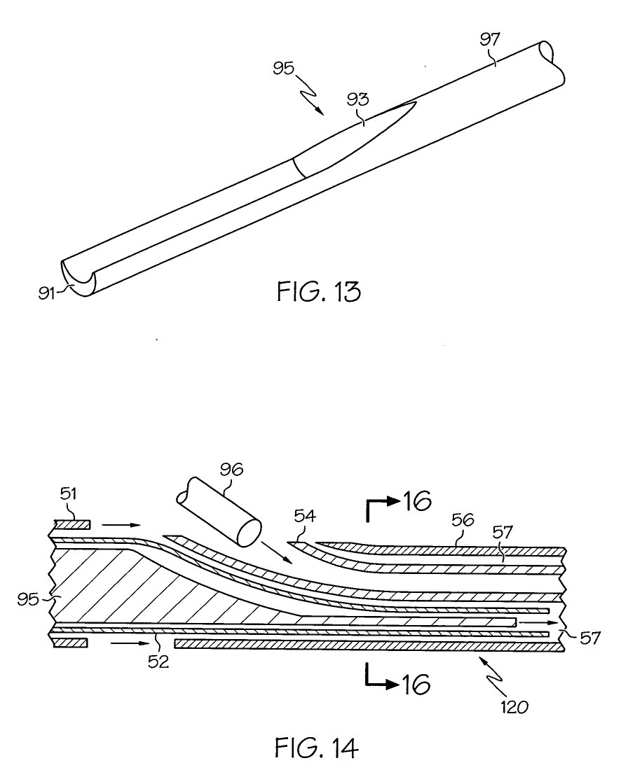

An exemplary crescent-shaped mandrel 98 such as that depicted in FIG. 15 has a crescent-shaped cross-section for a sufficient length of the mandrel 98 to maintain the inflation lumen's shape, particularly approaching the exchange joint's distal end 86 where the guidewire port 82 is larger and takes more space in the exchange joint 80. As best seen in FIG. 6, the inflation lumen 84 substantially consists of continuously formed first and second arced walls 85 and 87. The first wall 85 has a smaller radius of curvature than the second wall 87, and is arced to partially encircle the guidewire port 82. The outer surface 87 is also arced, and the two surfaces 85 and 87 create a crescent shape that gives the inflation lumen a large flow area while minimizing the exchange joint's longitudinal profile at the exchange joint distal end 86. Further, with the inflation lumen 84 gradually forming a crescent shape from the exchange joint proximal end 88 to the distal end 86 as the guidewire port 82 becomes increasingly entrenched in the exchange joint 80, the overall exchange joint is able to be formed with a small and uniform outer diameter. The mandrel 98 may have a crescent-shaped cross-section for all or most of the mandrel length. FIG. 13 is a perspective view of another exemplary mandrel 95 that has a crescent-shaped first end 91 and a stepped portion 93 that transitions the crescent shaped portion into a round portion 97. The stepped portion 93 and the round portion 97 support a substantial amount of the inflation lumen 84 when the exchange joint 80 is bonded to the guidewire lumen 54 and the distal shaft 92.

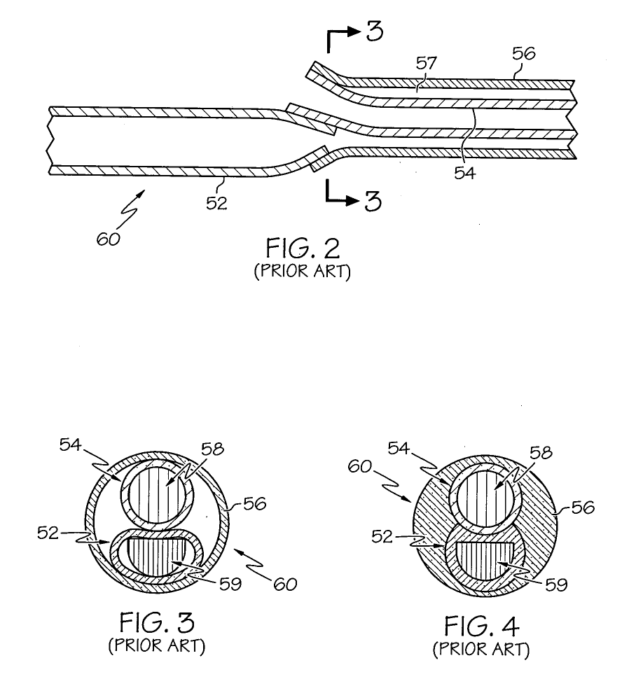

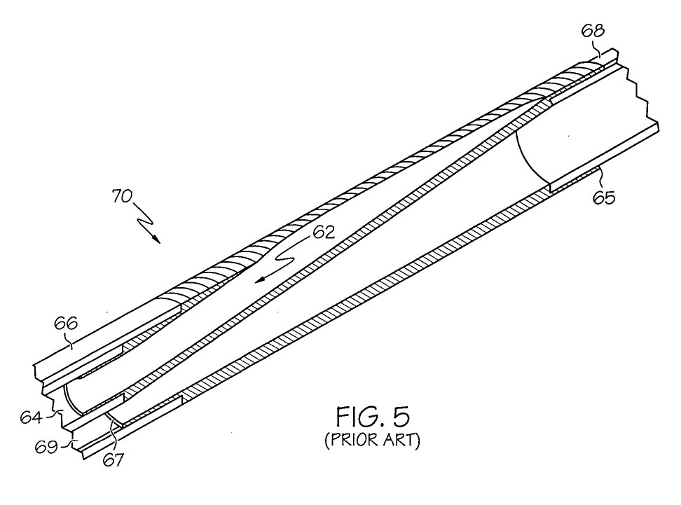

A crescent-shaped mandrel such as the mandrel 95 depicted in FIG. 13 is also useful when performing a multi-component exchange joint using a process similar to that previously discussed in connection with FIGS. 2 to 4. According to an exemplary method, an RX catheter exchange joint 120 depicted in FIG. 14 is assembled to include an elongate distal shaft 56 joined to transition tubing 52. The distal shaft 56 includes a coaxial inner guidewire lumen 54 extending to the shaft distal end. The transition tubing 52 joins the distal shaft 56 to a proximal shaft 51, which may optionally include or function as an inflation lumen through which a fluid is transported. FIG. 14 is a cross-sectional longitudinal view illustrating how the distal shaft 56 and the transition tubing 52 are joined. As depicted, the transition tubing 52 is inserted into the distal shaft 56. The guidewire lumen 54 is situated alongside the transition tubing at the position where the transition tubing 52 is inserted. During use, the transition tubing 52 transports fluid from the proximal shaft 51 to a distal shaft inflation lumen 57 that is coaxial with the guidewire lumen 54. Thus, the exchange joint 60 effectively transitions the inflation and guidewire lumens into the distal shaft 56 from a proximal side-by-side arrangement to a distal coaxial arrangement.

Assembly of the exchange joint 120 includes inserting the transition tubing 52 into the distal shaft 56. The inner diameter of the distal shaft 56 may need to be flared to allow room for the transition tubing 52, which also may require skiving. A round mandrel 96 is inserted into the lumen 54. Likewise, the crescent-shaped mandrel 95 is inserted into the transition tubing 52. FIG. 16 is a cross-sectional view of the exchange joint 120 taken along line 18-18 in FIG. 14 after performing a bonding procedure, with the crescent-shaped mandrel 95 loaded into the transition tubing 52 and the round mandrel 96 loaded into the guidewire lumen 54. The bonding process includes wrapping heat shrink material around the exchange joint 120. Heat is then applied to the exchange joint 120 as the heat shrink material compresses the joint components and brings the joint 120 to the bonded form depicted in FIG. 16. After the bonding process is completed, the mandrels 95 and 96 are removed and the lumen 54 is cut to form the guidewire entrance port.

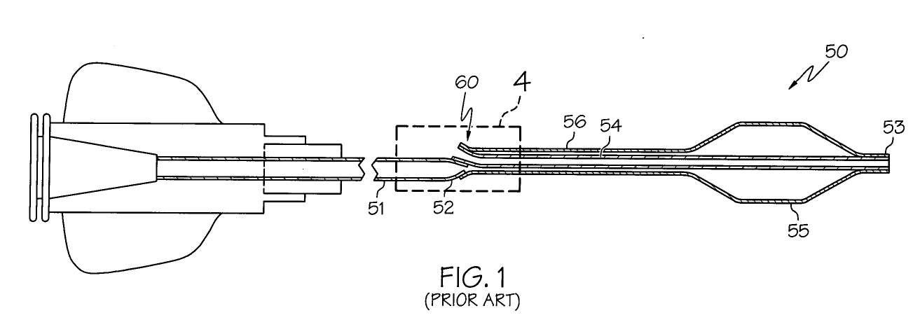

As previously discussed, the prior art RX catheter has an overall distinctively stepped shape at the exchange joint, as seen when viewing the joint 60 in FIG. 1. The step in the exchange joint 60 adds bulk to the shaft diameter, which may detrimentally affect catheter performance. More particularly, if the RX catheter 50 exits the distal end of a guide catheter, the step in the exchange joint 60 may become caught on the guide catheter edge while withdrawing the RX catheter from the vessel. Unlike the prior art assembly, the present exchange joint assembled using the crescent-shaped mandrel 95 has a substantially uniform outer diameter, as seen when viewing FIG. 14.

Using either of the above processes, an RX catheter having a unitary or a multi-component exchange joint may be manufactured. Each of the exchange joints includes an inflation lumen that transitions between a substantially round cross section to a crescent-shaped cross section in order to maintain a substantially uniform cross section from one end of the joint to the other. Although each joint provides different advantages, the unitary exchange joint 80 depicted in FIG. 6 provides the particular advantage of a ready-made joint that does not require flaring or skiving to combine the various lumens. Further, the unitary exchange joint 80 provides a convenient guidewire port 82 that gradually steers a guidewire toward and into the RX catheter distal end. The unitary exchange 80 joint may be a flexible component, and preferably has elasticity similar to that of both the proximal and distal shafts to which it is attached. Various moldable biocompatible polymers may be used to mold the unitary exchange joint 80 including polyamides, blends of polyamides and polyolefins, liquid crystal polymers, polyesters, polyketones, polyimides, polysulphones, polyoxymethylenes, polycarbonate, polymethyl methacrylate, polyolefins, cross-linked polyolefins, grafted polyolefins and other compatibilizers based on polyolefins. Lubrication additives may be included such as polyethylene micro-powders, fluoropolymers, silicone-based oils, fluoro-ether oils, molybdenum disulphide, graphite, and polyethylene oxide. Reinforcing additives may also be included, such as nano-clays, carbon fibers, and glass fibers or spheres. In addition, the unitary exchange joint 80 may be manufactured from harder and/or stiffer materials including biocompatible ceramics and biocompatible metals such as stainless steel.

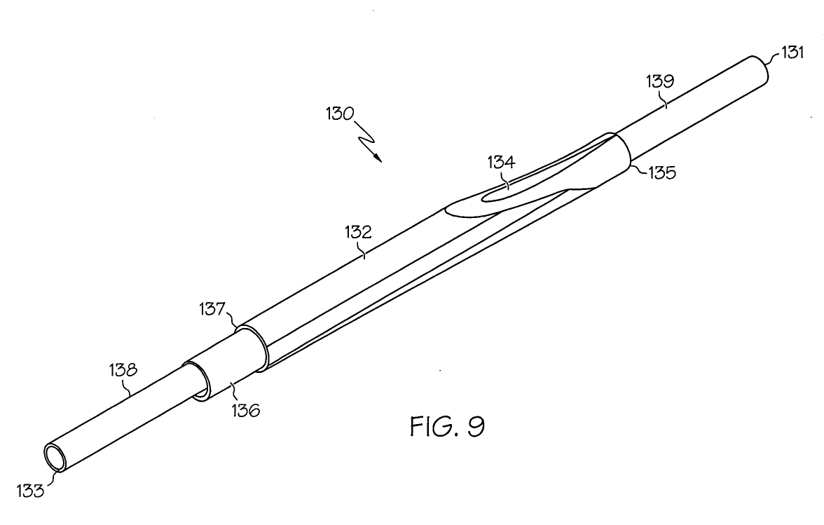

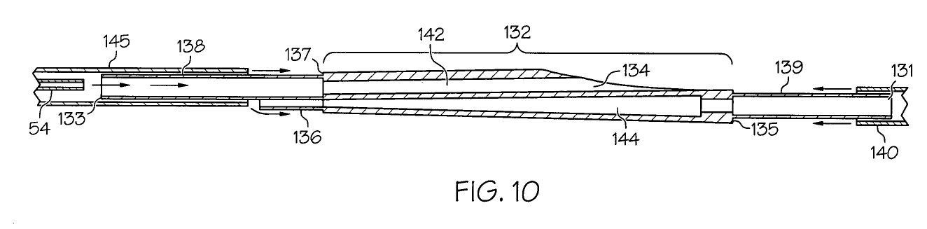

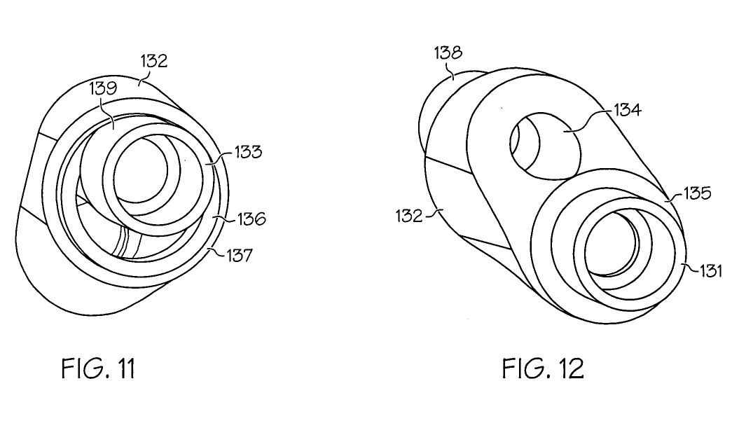

Turning now to FIGS. 9 to 12, another exemplary exchange joint 130 is depicted. FIGS. 9, 11, and 12 are perspective views of the exchange joint 130 at different angles. FIG. 11 is a perspective view of the exchange joint 130 taken from the joint distal end 133, and FIG. 12 is a perspective view taken from the joint proximal end 131. The exchange joint 130 comprises a main body portion 132 that includes a guidewire port 134, a guidewire lumen, and an inflation lumen 142 that transitions from having a substantially round cross-section to a crescent-shaped cross-section. At the exchange joint proximal end, an elongate tube 139 having a circular cross-section extends from the main body portion 132. As seen from viewing FIG. 10, the elongate tube 139 is in communication with the inflation lumen 142 and aids in attaching the RX catheter proximal shaft 140 to the exchange joint 130. To attach the two, the RX catheter proximal shaft 140 slides over the elongate tube proximal end 131 until the proximal shaft 140 abuts the main body portion proximal end 135.

At the exchange joint distal end, an inner lumen 138 and an outer lumen 136 extend from the main body portion 132. As seen from viewing FIG. 10, the inner lumen 138 slidingly receives a guidewire lumen 54. When properly inserted, the hypotube abuts the main body distal end 137, exits the inner lumen distal end 133, and extends to the RX catheter distal tip. During use, a guidewire is inserted into the guidewire port 134, through the main body guidewire lumen 144 and then into the guidewire lumen 54. The outer lumen 136 may be defined in part by tubular extension from the exchange joint 130 or, as depicted in FIG. 11, by an arced wall that is an extension of the crescent-shaped inflation lumen 142. The outer lumen 136 further aids in attaching the RX catheter distal shaft 145 to the exchange joint 130. To join the two, the RX catheter distal shaft 145 slides over the outer lumen 136 until the distal shaft 145 abuts the main body portion distal end 137.

In FIG. 10, the exchange joint 130 is depicted as a unitary assembly including the main body portion 132, the elongate tube 139, the inner lumen 138 and the outer lumen 136 molded as an integral exchange joint 130. However, the exchange joint 130 may also be assembled by manufacturing the elongate tube 139, the inner lumen 138 and the outer lumen 136 separately and then joining them together using a conventional process such as thermal bonding or UV cure bonding with adhesive. The exchange joint 130 may be a flexible component, and preferably has elasticity similar to that of both the proximal and distal shafts to which it is attached. Each component in the exchange joint 130 may be made using any of the materials previously listed with respect to the exchange joint 80 depicted in FIG. 6.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing the exemplary embodiment or exemplary embodiments. It should be understood that various changes can be made in the function and arrangement of elements without departing from the scope of the invention as set forth in the appended claims and the legal equivalents thereof.

What is claimed is:

1. A rapid exchange catheter, comprising:
an elongate proximal shaft comprising a proximal lumen;
an elongate distal shaft defining an distal outer lumen;
an elongate distal inner lumen inside the distal shaft; and
an exchange joint coupled between the elongate proximal shaft and the elongate distal shaft, and having proximal and distal ends, the exchange joint comprising:
a guidewire port providing external access for a guidewire into the elongate distal inner lumen, and
a transition lumen in communication with the proximal lumen and the distal outer lumen, the transition lumen having a crescent-shaped cross-section at least at the exchange joint distal end.

2. The rapid exchange catheter according to claim 1, wherein the transition lumen has a substantially round cross section at the exchange joint proximal end.

3. The rapid exchange catheter according to claim 1, wherein the transition lumen has a cross sectional shape that wraps partially around the guidewire port at least at the exchange joint distal end.

4. The rapid exchange catheter according to claim 1, wherein the exchange joint has a substantially uniform and circular outer diameter.

5. The rapid exchange catheter according to claim 1, wherein the exchange joint is formed from a flexible polymer.

6. The rapid exchange catheter according to claim 1, wherein the exchange joint is an integrally molded structure.

7. The rapid exchange catheter according to claim 1, wherein the guidewire port forms a gradually deepening trench in the exchange joint.

8. The rapid exchange catheter according to claim 6, wherein the exchange joint further comprises a surface defining the guidewire port, the surface having a C-shaped cross-section at the exchange joint distal end.

9. The rapid exchange catheter according to claim 1, further comprising:
an elongate tube extending from the exchange joint proximal end and in communication with the transition lumen, the elongate tube joining the elongate proximal shaft to the exchange joint.

10. The rapid exchange catheter according to claim 1, further comprising:
an elongate tube extending from the exchange joint distal end and in communication with the guidewire port, the elongate tube joining the elongate distal inner lumen to the exchange joint.

11. The rapid exchange catheter according to claim 10, further comprising:
an arced wall extending from the exchange joint distal end and, together with the elongate tube, defining an extension of the transition lumen, the extension of the transition lumen having a crescent-shaped cross-section, and the arced wall joining the elongate distal shaft to the exchange joint.

12. The rapid exchange catheter according to claim 1, wherein the transition lumen is an inflation lumen.

13. An exchange joint for joining a proximal shaft, a distal shaft, and an elongate distal inner lumen inside the distal shaft in a rapid exchange catheter, the exchange joint comprising:
a proximal end adapted to be coupled to the proximal shaft;
a distal end adapted to be coupled to the distal shaft;
a guidewire port providing external access for a guidewire into the elongate distal inner lumen, and
a transition lumen in communication with the proximal shaft and the distal shaft, the transition lumen having a substantially round cross section at the exchange joint proximal end, and a crescent-shaped cross-section at least at the exchange joint distal end.

14. The exchange joint according to claim 13, wherein the transition lumen has a cross sectional shape that wraps partially around the guidewire port at least at the exchange joint distal end.

15. The exchange joint according to claim 13, wherein the exchange joint has a substantially uniform and circular outer diameter.

16. The exchange joint according to claim 13, wherein the exchange joint is formed from a flexible polymer.

17. The exchange joint according to claim 13, wherein the exchange joint is an integrally molded structure.

18. The exchange joint according to claim 13, wherein the guidewire port forms a gradually deepening trench in the exchange joint.

19. The exchange joint according to claim 18, further comprising a surface defining the guidewire port, the surface having a C-shaped cross-section at the exchange joint distal end.

20. The exchange joint according to claim 13, further comprising:
a first elongate tube extending from the exchange joint proximal end and in communication with the transition lumen, the elongate tube joining the elongate proximal shaft to the exchange joint;
a second elongate tube extending from the exchange joint distal end and in communication with the guidewire port, the elongate tube joining the elongate distal inner lumen to the exchange joint; and
an arced wall extending from the exchange joint distal end and, together with the elongate tube, defining an extension of the transition lumen, the extension of the transition lumen having a crescent-shaped cross-section, and the arced wall joining the elongate distal shaft to the exchange joint.
